(19) United States
(12) Patent Application Publication
(10) Pub. No.: US 2023/0190358 A1
(43) Pub. Date: Jun. 22, 2023

(54) APPARATUS FOR DRIVING A SURGICAL END EFFECTOR

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 18/076,002

(22) Filed: Dec. 6, 2022

Related U.S. Application Data
(60) Provisional application No. 63/292,330, filed on Dec. 21, 2021.

(51) Int. Cl. A61B 18/14

(52) U.S. Cl. CPC A61B 18/14; A61B 2018/00601

(57) ABSTRACT

When surgical devices operate on tissue (e.g. cutting or ablating tissue), tissue and other debris is typically sucked away from the operating site along a suction pathway through the device. In some cases, this debris may accumulate at certain points within the device. This can lead to blockages forming in the suction pathway, which reduces the flow of debris through the device and hinders effective removal of debris from the operating site. The present invention addresses these issues by providing an apparatus which is configured to drive the end effector of a surgical instrument in an operational mode that causes tissue debris to be dislodged from the surgical instrument.

### TECHNICAL FIELD

Embodiments of the present invention described herein relate to an apparatus for driving a surgical end effector of a surgical instrument. In particular, embodiments of the present invention relate to an apparatus for driving a surgical end effector of a surgical instrument including a rotary shaver arrangement.

### BACKGROUND TO THE INVENTION AND PRIOR ART

Traditional mechanical surgical instruments utilise mechanical means for cutting tissue. Electrosurgical instruments provide advantages over such traditional surgical instruments in that they can be used for coagulation and tissue sealing purposes. One such prior art arrangement is known from U.S. Pat. No. 5,904,681, which describes a surgical instrument including a mechanical cutting portion, such as a rotary blade or burr, and a radio frequency (RF) cutting and/or cauterizing portion comprising an electrosurgical instrument which operates in bipolar mode. A rotary burr works best to remove hard tissues, such as bone, while the bipolar electrosurgical instrument can be used to cut or ablate soft tissues and/or cauterize tissue, including blood vessels. Alternatively, the mechanical cutting portion may include a rotary blade, which may be used for removing soft tissues, with the electrodes of the electrosurgical system being used for cauterisation or coagulation.

Another prior art arrangement is known from WO 2016/171963 A1, which describes an elongated shaft assembly including a rotatable inner cutting sleeve and a non-rotating outer sleeve. A window of the inner cutting sleeve is selectively rotatable within an opening of the non-rotating outer sleeve to cut tissue with a sharpened cutting blade when rotated in a first rotational direction and to cut tissue with an electrode when rotated in a second, opposite, rotational direction.

### SUMMARY OF THE INVENTION

When surgical devices operate on tissue (e.g. cutting or ablating tissue), tissue and other debris is typically sucked away from the operating site along a suction pathway through the device. In some cases, this debris may accumulate at certain points within the device. This can lead to blockages forming in the suction pathway, which reduces the flow of debris through the device and hinders effective removal of debris from the operating site.

In view of the above, from an aspect the present invention provides an apparatus for driving a surgical end effector of a surgical instrument, the apparatus comprising: drive componentry configured to drive the end effector to operate in use; and drive control circuitry configured to control the drive componentry to drive the end effector in a first operational mode to cause its intended surgical effect on tissue to be treated, and to drive the end effector in a second operational mode that causes tissue debris to be dislodged from the surgical instrument.

In some embodiments, the first operational mode causes the end effector to be driven at a first operational frequency, and the second operational mode causes the end effector to be driven at a second operational frequency different from the first operational frequency.

In some embodiments, the second operational frequency is greater than the first operational frequency.

In some embodiments, the drive control circuitry is configured to control the drive componentry to move the end effector to cause the debris to be dislodged from the surgical instrument.

In some embodiments, the drive componentry is configured to drive at least a part of the end effector to rotate and/or reciprocate in use.

In some embodiments, the apparatus is a hand-piece.

From an aspect the present invention provides a surgical device comprising: a surgical instrument comprising a surgical end effector; and an apparatus as set out above.

From an aspect the present invention provides a surgical system, comprising: a suction source; and a surgical device according as set out above.

In some embodiments, the apparatus is for driving an end effector of an RF rotary shaver surgical device. In an RF rotary shaver surgical device, mechanical tissue shaving functionality is combined with the tissue ablation effect of an RF electrode. This means that the suction pathway through the instrument and the handpiece must accommodate tissue debris from shaving as well as RF ablation use. In an RF shaver instrument and handpiece configuration where the majority of the RF shaver instrument is the same as a standard shaver instrument, the flow path of debris from the distal tip rearwards is identical to that of a standard shaver instrument and handpiece. In this case, the flow path through the instrument and handpiece is designed for a spinning inner hub, which is the mechanism used to connect the rotational torque generated by the motor to the disposable instrument. One drawback of this configuration for an RF rotary shaver instrument is that during RF ablation, there will be no movement of the inner shaver blade, and therefore also no movement of the inner hub. In its stationary position, the inner hub can act as a catch point for tissue, and with prolonged RF ablation of tissue, this can lead to reduced flow and clogging due to tissue debris build up in this area. This is not a problem for standard RF devices, because these do not have a flow restriction in this area. Most of these devices have a single suction tube all the way through the handpiece and out of the instrument. Therefore, the problem is particularly an issue for RF shavers with a ‘traditional’ motor to inner hub connection design and tissue flow over the inner hub.

Some embodiments of the present invention use a motor control algorithm to move a motor (and therefore the drive-bit and the inner hub) a small amount during RF ablation. For example, the motor may vibrate the drive-bit and the inner hub with very fast forward/backward oscillations of a very small angular displacement every few seconds, or as often as a de-clogging function is required. If clogging is a significant problem, the vibration could occur during every instance of ablation for as long as the ablation continues.

During RF ablation, the inner and outer blades of the shaver arrangement would usually be in a ‘parked’ configuration to allow all suction to be directed through the RF suction opening. Preferably, the size of the oscillations is kept small so that the inner shaver blade teeth are not exposed during RF ablation. If the blade window were to open, this could potentially cause unwanted collateral tissue damage.

The timing, amplitude, acceleration and any other relevant characteristics of the oscillation of the motor could be tuned to best shake any debris off the inner hub and out of the inner shaft as a whole before the flow path becomes fully clogged and the suction functionality of the device in either RF or shave modes is impaired. The mechanism could also be used to clear clogs occurring at the distal tip during RF use, if tissue debris formed during ablation were to get stuck on the teeth of the inner blade or the distal end of the inner shaft opening.

In view of the above, from an aspect the present invention provides an apparatus for driving a rotary shaver arrangement of a surgical instrument, the apparatus comprising: drive componentry configured to drive the rotary shaver arrangement to operate in use; drive control circuitry configured to control the drive componentry to drive the rotary shave arrangement to alternate between rotation in a first rotational direction and rotation in a second rotational direction opposite to the first rotational direction at a frequency sufficient to dislodge debris from the surgical instrument.

In some embodiments, the drive control circuitry is configured to control the drive componentry to drive the rotary shave arrangement such that, when alternating between rotation in the first rotational direction and rotation in the second rotational direction, the angular displacement of the rotary shave arrangement is limited to a predetermined range of angular displacement values.

In some embodiments, the apparatus comprises a user-operable button. The drive control circuitry is configured to control the drive componentry to drive the rotary shave arrangement to alternate between rotation in the first rotational direction and rotation in the second rotational direction in response to the button being operated by the user.

In some embodiments, the drive control circuitry is configured to control the drive componentry to drive the rotary shave arrangement to alternate between rotation in the first rotational direction and rotation in the second rotational direction for a predetermined time after the button is operated by the user.

In some embodiments, the apparatus comprises RF electrical connections configured to supply an RF signal to an electrode of the surgical instrument,

In some embodiments, the drive control circuitry is configured to control the drive componentry to drive the rotary shave arrangement to alternate between rotation in the first rotational direction and rotation in the second rotational direction while the apparatus supplies an RF signal to the electrode through the RF electrical connections.

In some embodiments, the drive componentry comprises a portion configured to engage with an end portion of the surgical instrument, the end portion being connected to the rotary shaver arrangement.

In some embodiments, the apparatus is a hand-piece.

From an aspect the present invention provides a surgical device comprising: a surgical instrument comprising a rotary shaver arrangement; and an apparatus as set out above.

In some embodiments, the rotary shaver arrangement has a rotatable tubular element with a cutting portion having a cutting blade formed therein that when in use is able to cut tissue.

In some embodiments, the interior of the rotatable tubular element in use acts as a suction lumen to permit the aspiration of debris during use.

In some embodiments, the surgical instrument comprises an active electrode.

From an aspect the present invention provides a surgical system, comprising: a suction source; and a surgical device as set out above.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a schematic diagram of a surgical system including a surgical device according to an embodiment of the present invention;

FIG. 2 is a side view of a surgical device according to an embodiment of the present invention;

FIG. 3 is a perspective view of an example of the tip shown in FIG. 2, wherein the rotating shaver blade's cutting edges are facing upwards;

FIG. 4 is a perspective view of the tip shown in FIG. 3, wherein the rotating shaver blade's cutting edges are facing downwards (cutting edges cannot be seen);

FIG. 5 is a plan view of the tip shown in FIG. 3, wherein the rotating shaver blade's cutting edges are facing upwards;

FIG. 6 is a side view of the tip shown in FIG. 3, wherein the rotating shaver blade's cutting edges are facing upwards;

FIG. 7 is a side view of the tip shown in FIG. 3, wherein the rotating shaver blade's cutting edges are facing downwards;

FIG. 8 is an end view of the tip shown in FIG. 3, wherein the rotating shaver blade's cutting edges are facing upwards;

FIG. 9 is a perspective view of another example of the tip shown in FIG. 2, wherein the rotating shaver blade's cutting edges are facing upwards;

FIG. 10 is a perspective view of the tip shown in FIG. 9, wherein the rotating shaver blade's cutting edges are facing downwards (cutting edges cannot be seen);

FIG. 11 is a plan view of the tip shown in FIG. 9, wherein the rotating shaver blade's cutting edges are facing upwards;

FIG. 12 is a side view of the tip shown in FIG. 9, wherein the rotating shaver blade's cutting edges are facing upwards;

FIG. 13 is a side view of the tip shown in FIG. 9, wherein the rotating shaver blade's cutting edges are facing downwards;

FIG. 14 is an end view of the tip shown in FIG. 9, wherein the rotating shaver blade's cutting edges are facing upwards;

FIG. 15 is a side view of a surgical instrument according to an embodiment of the present invention;

FIG. 16 is a perspective view of the inner hub of FIG. 15; and

FIGS. 17A and 17B are perspective views showing the rotating shaver blade in different rotational positions.

### DESCRIPTION OF THE EMBODIMENTS

An apparatus is described herein in the context of a surgical device for performing procedures such as ablation, sealing, resection and coagulation of tissues. The surgical device is described herein by way of example as an electrosurgical device.

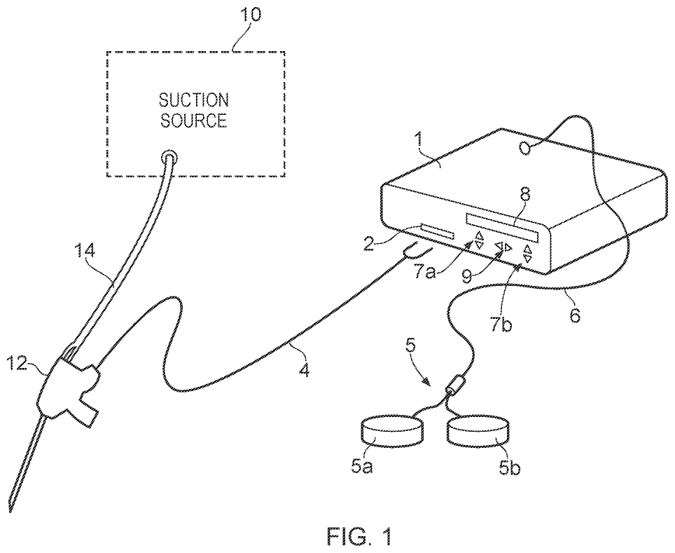

Referring to the drawings, FIG. 1 shows an electrosurgical generator 1 having an output socket 2 providing a radio frequency (RF) output, via a connection cord 4, for an electrosurgical device 12. The device 12 has a suction tube 14 which is connected to a suction source 10. Activation of the generator 1 may be performed from the device 12 via a handswitch (not shown) on the device 12, or by means of a footswitch unit 5 connected separately to the rear of the generator 1 by a footswitch connection cord 6. In the embodiment shown in FIG. 1, the footswitch unit 5 has two footswitches 5a and 5b for selecting a coagulation mode or a cutting or vaporisation (ablation) mode of the generator 1 respectively, although in some embodiments of the electrosurgical device 12 described herein it is envisaged that only one or other of the coagulation or ablation modes would be used, with cutting being provided mechanically by way of a rotating tube having a sharpened cut-out portion, described further below. In other embodiments, the cutting may be provided mechanically by way of a reciprocating cutting blade. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

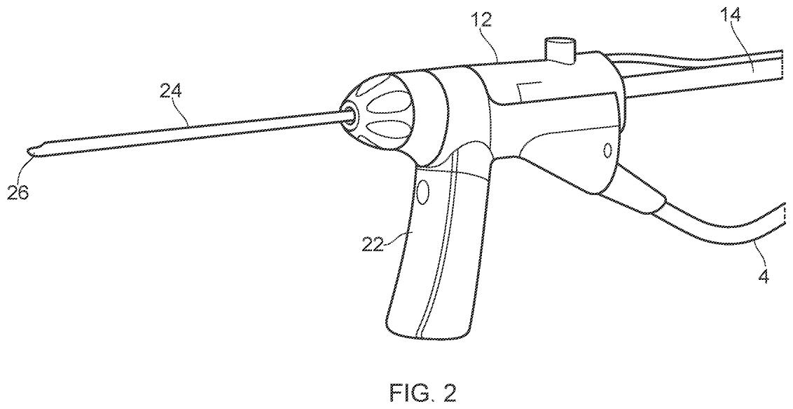

FIG. 2 shows a surgical device 12 according to an embodiment of the present invention. In the present embodiment, the surgical device is an electrosurgical device. The device 12 includes drive componentry (not shown), drive control circuitry (not shown) and a proximal handle portion 22. The device 12 also includes a surgical instrument including a hollow shaft 24 extending in a distal direction away from the proximal handle portion, and a distal end effector assembly 26 at the distal end of the outer shaft. A power connection cord 4 connects the device to the RF generator 1, whereas tubes 14 connect the device to the suction source 10. The device may further be provided with activation buttons (not shown), to allow the surgeon operator to activate either the mechanical cutting function of the end effector (which may be, for example, a rotary cutting function, a reciprocating cutting function or a combined rotary and reciprocating cutting function), or the electrosurgical functions of the end effector, which in this embodiment typically comprise coagulation or ablation.

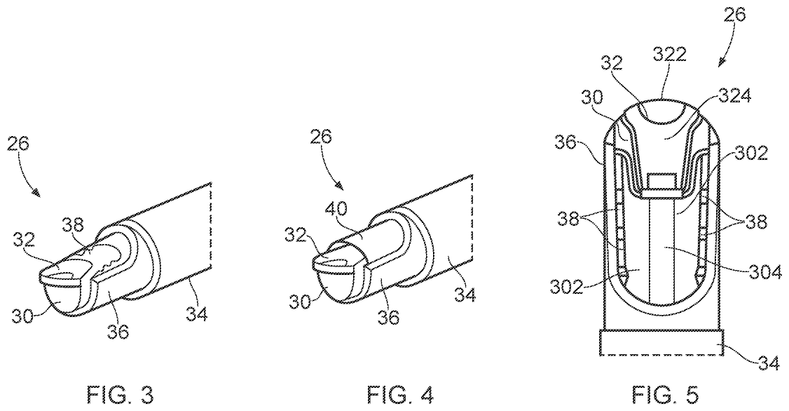

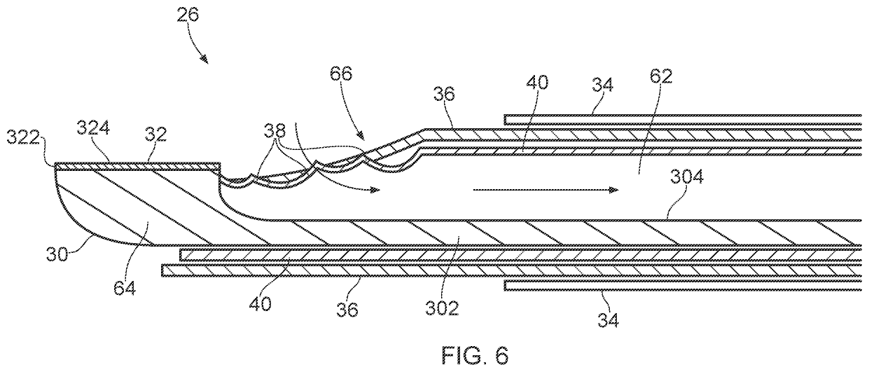

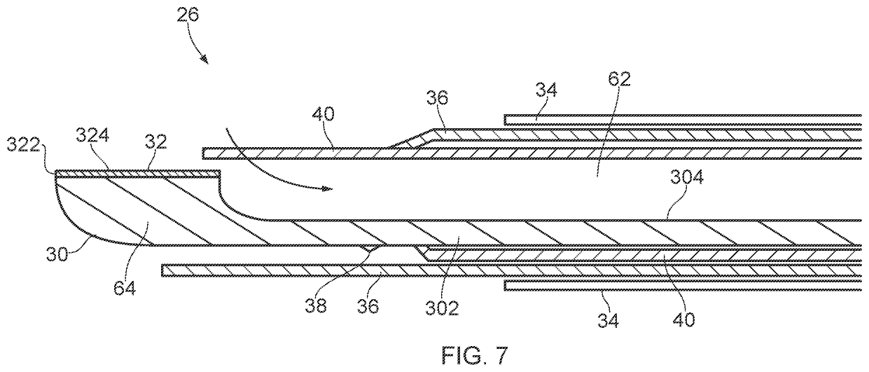

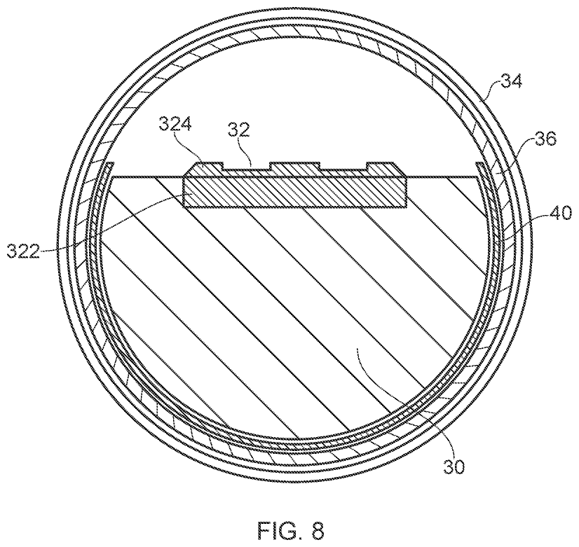

FIGS. 3 to 8 show an example of the distal end effector assembly 26 shown in FIG. 2 in more detail. In this example, the end effector assembly comprises a rotary shaver arrangement. With specific reference to FIGS. 6 and 7, the end effector comprises a series of concentrically arranged tubes, with outer insulating sheath 34 containing a hollow conductive tube 36, having at its distal end an open end and an opening cut out of one side thereof to act as a cutting window 66. The edges of the cutting window 66 may be sharpened to provide scissor action in use against a cutting edge 38 of a cylindrical shaver element 40 which is rotatable about a longitudinal axis of the instrument. The hollow, distally open-ended conductive tube 36 acts as a return electrode and concentrically surrounds a rotatable cylindrical shaver element 40. By ‘concentrically surrounds’ we mean that the shaver element 40 is inside and coaxial with the tube 36. The proximal part of the tube 36 is covered with the insulating sheath 34. The distal part of the tube 36 has the opening which acts as the cutting window 66. The shaver blade itself is a hollow cylinder of C-shape cross-section at the distal end, meaning a hollow cylinder which has a segment cut out for a portion of the distal end, so that the cylinder is incomplete at the distal end. The cut out portion is sharpened and serrated, to form the cutting edge 38.

At the distal end of the end effector, the shaver blade 40 has a sharp cutting edge 38, which may be serrated or shaped into points to provide cutting teeth. The hollow open-ended shaver blade 40 in use defines an internal suction lumen 62, which extends along the shaft 24 and ultimately connects to the suction source 10. Disposed within the hollow shaver blade 40 is a pipe-shaped insulating ceramic body 30. The ceramic body 30 comprises an elongate stem portion 302, and an upturned head portion 64 which extends out of the open end of the shaver blade 40 beyond the distal end of the shaver blade 40 and the shaft 36. Mounted on an upper plane surface 322 of the ceramic body 30 is the active electrode 32. The active electrode 32 has a portion 304 which runs along ceramic stem portion 302 back to the generator 1 via the connection cord 4. A significant planar portion 324 of the active electrode 32 mounted on the head of the ceramic 64 faces upwards in the same direction as the cutting edges 38. A smaller portion of the active electrode 322 is forward facing, constituting the distal-most part of the active electrode 32. Effectively, however, the planar portion 324 faces orthogonally to the longitudinal axis of the instrument, and is effective in the same direction as the shaver blade 40. That is, the shaver blade 40 is operative when in use to cut tissue that it is presented against and which is located in a direction to the side of the shaft of the instrument i.e. in a direction orthogonal to the longitudinal axis of the instrument. The active electrode 32, due to the large planar face 324 also operatively faces in the same direction as the operative direction of the shaver blade 40, so that in use it may coagulate or ablate tissue that is also in the same direction to the side of the shaft of the instrument i.e. in the same orthogonal direction to the longitudinal axis of the instrument as the operative direction of the shaver blade, depending on the operating mode.

In some embodiments, the use of the shaver blade 40 and the electrode 32 can be simultaneous. This is possible as the described example is an open ended tubular design, which still has bidirectional cutting edges 38. This allows a significant portion of the electrode 324 to be positioned facing the same direction (upwards) as the cutting window 66. This means the electrode 32 and the shaver blade 40 can operate on the same section of tissue simultaneously, such that both mechanical cutting of the tissue (using the blade 40), and electrosurgical coagulation or ablation of the tissue (using the active electrode 32) can be obtained simultaneously.

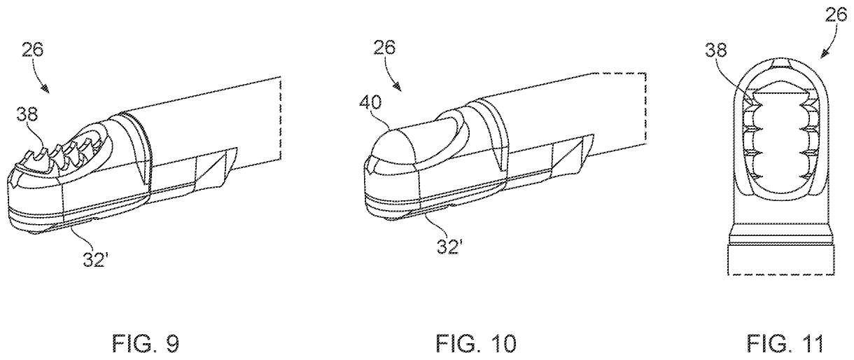

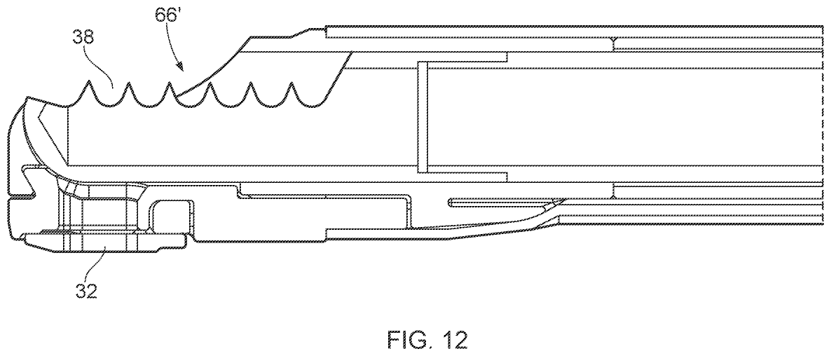

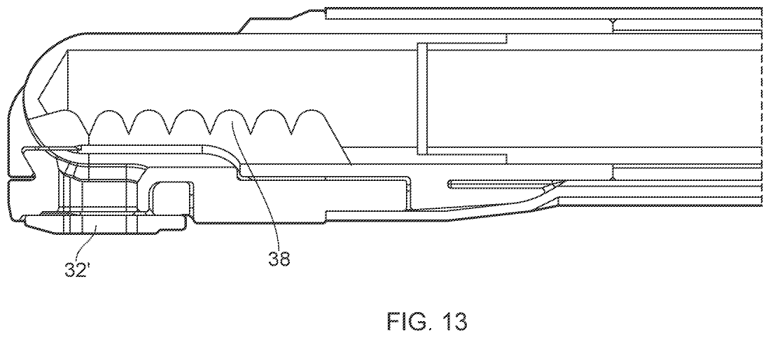

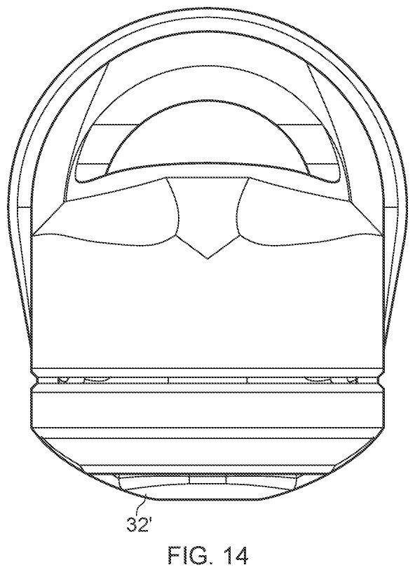

FIGS. 9 to 14 show another example of the distal end effector assembly 26 shown in FIG. 2. This example is similar to the example described in relation to

FIGS. 3 to 8. However, in this example, the active electrode 32′ and cutting window 66′ are on opposite sides of the assembly.

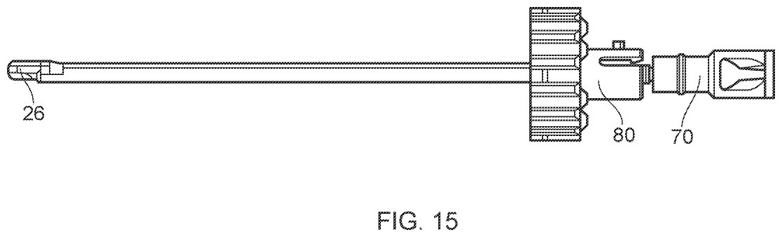

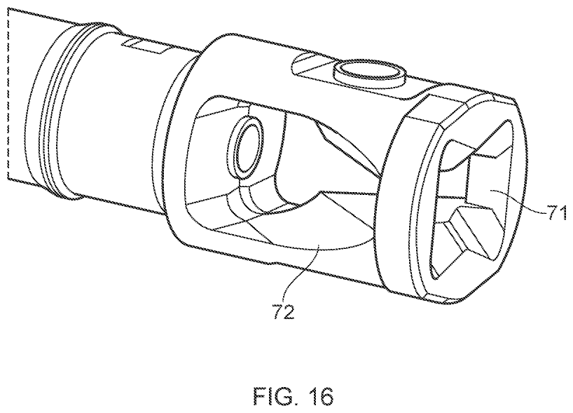

FIGS. 15 and 16 show an inner hub 70 which is located at a proximal end of the surgical instrument shown in FIG. 2. The inner hub 70 is connected to the shaver blade, such that rotation of the inner hub 70 causes rotation of the shaver blade in use. The inner hub 70 includes a magnet (not shown), which allows the surgical device 12 to determine the orientation of the hub using a Hall sensor. This allows the system to rotate the blade to the closed position. The surgical instrument also includes an outer hub 80, which remains static during use.

The drive componentry of the surgical device includes a motor (not shown) and a drive-bit (not shown) connected to the motor. The drive-bit defines a rotational axis which is substantially parallel to the longitudinal axis of the instrument. As shown in FIG. 16, the inner hub 70 contains a void 71 which is arranged to receive the drive-bit of the surgical device. When inserted into the inner hub 70, the rotation of the drive-bit with the motor drives the inner hub 70 to rotate, thereby causing the shaver blade to rotate.

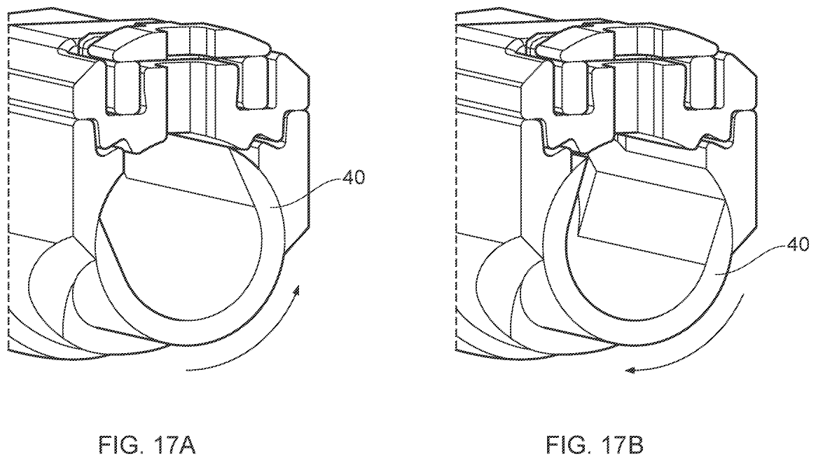

The inner hub 70 includes projecting features 72 which may catch tissue or other debris when the inner hub 70 is stationary. This may lead to obstruction of tissue and liquid flow through the inner hub 70. In order to address this issue, the device includes drive control circuitry (not shown) which is configured to control the motor to drive the inner hub 70 to alternate between rotation in a first rotational direction and rotation in a second rotational direction opposite to the first rotational direction with respect to the rotational axis defined by the drive-bit. This alternating rotation of the inner hub 70 may be referred to herein as oscillation of the inner hub 70. The alternating rotation of the inner hub 70 causes corresponding rotation of the shaver blade 40, as illustrated in FIGS. 17A and 17B.

The drive control circuitry is configured to control the motor to drive the inner hub to oscillate with a particular frequency which is such that any tissue which may accumulate on features 72 of the inner hub 70 during use is dislodged by the oscillation. For example, the motor may drive the inner hub to oscillate with a frequency of at least 5 Hz. The motor may drive the inner hub to oscillate with a frequency in the range 5 Hz to 500 Hz, 10 Hz to 500 Hz, or 50 Hz to 500 Hz. In addition, the corresponding oscillation of the shaver blade allows tissue accumulated on the cutting edge of the shaver blade, or the opening at the distal end of the tube, to be dislodged.

In some embodiments, the drive control circuitry is configured to control the motor such that, when alternating between rotation in the first rotational direction and rotation in the second rotational direction, the angular displacement of the shaver blade is limited to a predetermined range of angular displacement values. For example, the angular displacement of the shaver blade may be limited to ±10°, ±20°, ±30° or ±40° with respect to the blade closed position. This limitation ensures that the teeth of the shaver blade are not exposed during oscillation of the shaver blade, avoiding the chance of unwanted collateral tissue damage.

In some embodiments, the oscillation of the inner hub may be triggered by the surgeon operator pressing an activation button (not shown). In such embodiments, the motor may drive the inner hub to continue oscillating for as long as the activation button is held down, stopping the driving of the inner hub once the activation button is released. In other such embodiments, the motor may drive the inner hub to oscillate for a predetermined time (e.g. 5 to 10 seconds) after the button has been released.

Referring again to FIGS. 3 to 14, to electrosurgically ablate or coagulate tissue, the user manipulates the device 12 such that the active electrode 32, 32′ is adjacent to the tissue to be treated, and activates the generator 1 to supply RF power to the active electrode 32, 32′, via the connection cord 4 and RF electrical connections within the device (not shown). The RF signal supplied is dependent on whether the active electrode is to simply coagulate (dessicate) tissue, or to ablate the tissue, wherein a higher power RF signal is used for tissue ablation than tissue coagulation. The active electrode 32, 32′ and the return electrode 36 act in a bipolar electrode arrangement. The suction lumen 62 is connected to the suction source 10 such that fluid, tissue fragments, bubbles or other debris in the vicinity of the electrode 32, 32′ can be aspirated from the surgical site. In some embodiments, the drive control circuitry is configured to control the motor to drive the inner hub to oscillate as long as an RF ablation signal is being supplied through the RF electrical connections. This constant oscillation prevents tissue or other debris generated by RF ablation from accumulating on the inner hub and/or the shaver blade during use.

To mechanically cut tissue, the user manipulates the instrument 12 such that the cutting window 66, 66′ is in contact with the tissue to be cut. The drive control circuitry controls the motor to drive the shaver blade 40 to rotate in a first rotational direction to cut tissue engaged by the cutting edges 38 on one side of the shaver blade 40, typically by shearing with an opposed edge on the cutting window 66 of the shaft 36. The drive control circuitry can also control the motor to drive the shaver blade to rotate in a second rotational direction opposite to the first rotational direction to cut tissue engaged by the cutting edges 38 on the other side of the shaver blade 40.

Various modifications whether by way of addition, deletion, or substitution of features may be made to above described embodiment to provide further embodiments, any and all of which are intended to be encompassed by the appended claims.

1. An apparatus for driving a surgical end effector of a surgical instrument, the apparatus comprising:
drive componentry configured to drive the end effector to operate in use; and
drive control circuitry configured to control the drive componentry to drive the end effector in a first operational mode to cause its intended surgical effect on tissue to be treated, and to drive the end effector in a second operational mode that causes debris to be dislodged from the surgical instrument.

2. An apparatus according to claim 1, wherein the first operational mode causes the end effector to be driven at a first operational frequency, and the second operational mode causes the end effector to be driven at a second operational frequency different from the first operational frequency.

3. An apparatus according to claim 2, wherein the second operational frequency is greater than the first operational frequency.

4. An apparatus according to claim 1, wherein the drive control circuitry is configured to control the drive componentry to move the end effector to cause the debris to be dislodged from the surgical instrument.

5. An apparatus according to claim 1, wherein the drive componentry is configured to drive at least a part of the end effector to rotate and/or reciprocate in use.

6. An apparatus according to claim 1, wherein the apparatus is a hand-piece.

7. An apparatus according to claim 1, wherein the apparatus is part of a surgical device comprising a surgical instrument with a surgical end effector.

8. An apparatus according to claim 1, wherein the apparatus is part of a surgical system, comprising a suction source; and a surgical instrument with a surgical end effector.

9. An apparatus for driving a rotary shaver arrangement of a surgical instrument, the apparatus comprising:
drive componentry configured to drive the rotary shaver arrangement to operate in use;
drive control circuitry configured to control the drive componentry to drive the rotary shave arrangement to alternate between rotation in a first rotational direction and rotation in a second rotational direction opposite to the first rotational direction at a frequency sufficient to dislodge debris from the surgical instrument.

10. An apparatus according to claim 9, wherein the drive control circuitry is configured to control the drive componentry to drive the rotary shave arrangement such that, when alternating between rotation in the first rotational direction and rotation in the second rotational direction, the angular displacement of the rotary shave arrangement is limited to a predetermined range of angular displacement values.

11. An apparatus according to claim 9, wherein the apparatus comprises a user-operable button,
wherein the drive control circuitry is configured to control the drive componentry to drive the rotary shave arrangement to alternate between rotation in the first rotational direction and rotation in the second rotational direction in response to the button being operated by the user.

12. An apparatus according to claim 11, wherein the drive control circuitry is configured to control the drive componentry to drive the rotary shave arrangement to alternate between rotation in the first rotational direction and rotation in the second rotational direction for a predetermined time after the button is operated by the user.

13. An apparatus according to claim 9, comprising RF electrical connections configured to supply an RF signal to an electrode of the surgical instrument,
wherein the drive control circuitry is configured to control the drive componentry to drive the rotary shave arrangement to alternate between rotation in the first rotational direction and rotation in the second rotational direction while the apparatus supplies an RF signal to the electrode through the RF electrical connections.

14. An apparatus according to claim 9, wherein the drive componentry comprises a portion configured to engage with an end portion of the surgical instrument, the end portion being connected to the rotary shaver arrangement.

15. An apparatus according to claim 9, wherein the apparatus is a hand-piece.

16. A surgical system, comprising:
a surgical instrument comprising a rotary shaver arrangement; and
a drive apparatus for driving the rotary shaver arrangement of the surgical instrument, the drive apparatus comprising:
drive componentry configured to drive the rotary shaver arrangement to operate in use;
drive control circuitry configured to control the drive componentry to drive the rotary shave arrangement to alternate between rotation in a first rotational direction and rotation in a second rotational direction opposite to the first rotational direction at a frequency sufficient to dislodge debris from the surgical instrument.

17. A surgical system according to claim 16, wherein the rotary shaver arrangement has a rotatable tubular element with a cutting portion having a cutting blade formed therein that when in use is able to cut tissue.

18. A surgical system according to claim 17, wherein the interior of the rotatable tubular element in use acts as a suction lumen to permit the aspiration of debris during use.

19. A surgical system according to claim 16, wherein the surgical instrument comprises an active electrode.

20. A surgical system according to claim 16, and further comprising a suction source.
